(19) United States
(12) Patent Application Publication
(10) Pub. No.: US 2024/0180995 A1
(43) Pub. Date: Jun. 6, 2024

(54) NUTRACEUTICAL COMPOSITION AND METHOD FOR THE PROTECTION OF HUMAN ARTERIAL ENDOTHELIAL CELLS FROM ENDOTHELIAL CELL DYSFUNCTION

(71) Applicant: K.L.R.M., LLC, San Pedro, CA (US)

(72) Inventor: Jacob Rajfer, San Pedro, CA (US)

(21) Appl. No.: 18/514,609

(22) Filed: Nov. 20, 2023

Related U.S. Application Data
(60) Provisional application No. 63/384,565, filed on Nov. 21, 2022.

(51) Int. Cl. A61K 36/9068; A61K 31/198; A61K 36/185; A61K 36/77; A61P 9/00

(52) U.S. Cl. CPC A61K 36/9068; A61K 31/198; A61K 36/185; A61K 36/77; A61P 9/00

(57) ABSTRACT

A method for protecting human arterial endothelial cells from endothelial cell dysfunction. The method includes administering a pharmaceutically effective amount of a composition over a sufficient period of time to stimulate NOS and protecting human arterial endothelial cells from endothelial cell dysfunction.

### CROSS REFERENCE TO RELATE APPLICATION

The present application claims the benefit of U.S. Provisional Application Ser. No. 63/384,565, entitled “NUTRACEUTICAL COMPOSITION AND METHOD FOR THE PROTECTION OF HUMAN ARTERIAL ENDOTHELIAL CELLS FROM ENDOTHELIAL CELL DYSFUNCTION,” filed Nov. 21, 2022, which is incorporated herein by reference.

### BACKGROUND OF THE INVENTION

### 1. Field of the Invention

The present invention relates to a nutraceutical composition and method for the protection of human arterial endothelial cells involved in the development of endothelial cell dysfunction.

### 2. Description of the Related Art

The arterial endothelium is an essential modulator of cardiovascular function. New Therapeutic Implications of Endothelial Nitric Oxide Synthase (eNOS) Function/Dysfunction in Cardiovascular Disease. Andreas Daiber, Ning Xia, Sebastian Steven, Matthias Oelze, Alina Hanf, Swenja Kröller-Schön, Thomas Münzel, Huige Li, Int J Mol Sci. 2019 January; 20(1): 187. Published online 2019 Jan. 7. doi: 10.3390/ijms20010187.PMCID: PMC6337296. Endothelial cells contribute to the response of the blood vessels to different physiological and pathological stimuli. Abnormalities in the endothelial cell structure and function may lead to cardiovascular diseases. New Therapeutic Implications of Endothelial Nitric Oxide Synthase (eNOS) Function/Dysfunction in Cardiovascular Disease. Andreas Daiber, Ning Xia, Sebastian Steven, Matthias Oelze, Alina Hanf, Swenja Kröller-Schön, Thomas Münzel, Huige, Li Int J Mol Sci. 2019 January; 20(1): 187. Published online 2019 Jan. 7. doi: 10.3390/ijms20010187.PMCID: PMC6337296; Cannon RO 3rd. Role of nitric oxide in cardiovascular disease: focus on the endothelium. Clin Chem. 1998 August; 44(8 Pt 2):1809-19. Erratum in: Clin Chem 1998 September; 44(9):2070. PMID: 9702990.

As those skilled in the art appreciate, the arterial endothelium is the innermost layer of the arterial blood vessel. The arterial endothelium is composed of cells that produce NO, a molecule required for normal endothelial function which includes vascular dilatation, platelet aggregation, and protection of the arterial vessel against injuries. Endothelial dysfunction (EnD), on the other hand, is defined as an inadequate production and/or release of NO from the arterial vascular endothelium that can result in a) an impairment in arterial vasodilation, b) migration and proliferation of the underlying vascular smooth muscle cells, and c) promotion of thrombosis due to platelet aggregation. When present, EnD has been heralded as an early event for the development of cardiovascular disease. IL-8 and PAI-1 are two inflammatory markers that rise in the blood when EnD) is present. In addition, flow mediated dilation (FMD)), a validated non-invasive test to measure vasodilator function of the peripheral vasculature, is a commonly used method for assessment of EnD).

### SUMMARY

In one aspect a method is provided for protecting human arterial endothelial cells from endothelial cell dysfunction. The method includes administering a pharmaceutically effective amount of a composition over a sufficient period of time to stimulate NOS and protecting human arterial endothelial cells from endothelial cell dysfunction.

In some embodiments the composition comprises ginger or a ginger derivative, Muira puama, Paullinia cupana, and L-arginine and/or L-citrulline.

In some embodiments the composition comprises 250 mg to 2 g ginger or ginger derivative.

In some embodiments the composition comprises 10 mg to 3 g of L-arginine, L-citrulline, or a mixture of L-arginine and L-citrulline.

In some embodiments the composition comprises 10 mg to 2 g of L-arginine, L-citrulline, or a mixture of L-arginine and L-citrulline.

In some embodiments the composition comprises 100 mg to 3 g of Muira puama.

In some embodiments the composition comprises 500 mg to 1.5 g of Muira puama.

In some embodiments the composition comprises at least 250 mg of Paullinia cupana.

In some embodiments the composition comprises 500 mg of Paullinia cupana.

In some embodiments the composition comprises 250 mg to 2 g of ginger or ginger derivative, 250 mg to 2 g of L-arginine, L-citrulline, or mixture of L-arginine and L-citrulline, 500 mg to 1.5 g of Muira puama, and 500 mg of Paullinia cupana.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic showing the role of the present nutraceutical composition (referred to below as COMP-4) in the protection of human arterial endothelial cells from endothelial dysfunction.

FIGS. 2A and 2B show the effect of the incubation of COMP-4 on cGMP (Cyclic guanosine monophosphate) expression in human umbilical arterial endothelial cells (HUAEC). FIG. 2A shows HUAEC cells were incubated for 24 hours with vehicle (CONTROL), the phosphodiesterase inhibitor IBMX (3-isobutyl-1-methylxanthine) as a positive control for cGMP expression, L-NAME (L-N^{G}-Nitro arginine methyl ester) as a non-selective inhibitor of the three nitric oxide synthases, and L-NIL (N⁶-(1-Iminoethyl)-lysine, hydrochloride) as a selective inhibitor of iNOS (inducible nitric oxide synthase). Afterward, cGMP was measured by colorimetric ELISA (enzyme-linked immunosorbent assay). *p<0.05, ***p<0.00 vs. control. FIG. 2B shows HUAEC cells were incubated for 24 hours with or without COMP-4 (0.69 mg/ml), as well as co-incubation of COMP-4 with L-NAME and L-NIL. Results for cGMP quantitation are expressed as pmol/mg protein and represent the mean+S.E.M. (standard error of the mean) of four duplicated experiments. **p<0.01; ***p<0.001 vs. control; #p<0.05; ##p<0.01 with respect to COMP-4.

FIGS. 3A, 3B, 3C, and 3D show the effect of the incubation of COMP-4 on nitric oxide (NO) formation in HUAEC. FIG. 3A shows nitrite production in the cell culture media. HUAEC were incubated for 24 hours with or without COMP-4. The cell culture media were collected and frozen at −80C. Nitrite formation was determined by the Griess reaction. Results are expressed as nM and represent the mean+S.E.M. of four experiments done in duplicates. ***p<0.001. FIG. 3B shows intracellular NO detection by confocal microscopy using a DAF-FM (Diaminofluorescein-FM) probe. HUAEC were incubated with or without COMP-4 for 4 hours and 24 hours. Fluorescence signal (shown in white markings) represents DAF-FM DA (Diaminofluorescein-FM diacetate). Counterstain is DAPI (4,6-diamidino-2-phenylindole). Magnification 200×. FIG. 3C shows intracellular detection of NO by flow cytometry using a DAF-FM probe. HUAEC cells were incubated with COMP-4 for 24 hours. LPS (lipopolysaccharide) and the NO donor, DETA NONOate (3,3-Bis(aminoethyl)-1-hydroxy-2-oxo-1-triazene), were used as positive controls of NO formation. Results are expressed as % of DAF-2 (Diaminofluorescein-2) positive live cells and represent the mean+S.E.M of two experiments done in duplicates. *p<0.05; **p<0).01 vs. control. Flow cytometry results are also shown for CONTROL, LPS, NO DONOR and COMP-4 with Side Scatter Area (SSC-A) as a function of Fluorescein Isothiocyanate (FITC)-A measurements. FIG. 3D shows intracellular detection of NO by the MuseR Nitric Oxide Kit. HUAEC cells were incubated with COMP-4. The kit uses DAX-J2™ (nitric oxide (NO) sensor developed by AAT Bioquest) Orange reagent to detect NO formation along with a marker of cell death, 7-AAD (7-Aminoactinomycin D). HUAEC were incubated with or without COMP-4. LPS was used as a positive control of NO formation. L-NAME was used as a non-selective inhibitor of NOS and co-incubated with COMP-4 for 24 hours. Results are expressed as % of DAX-J2™ positive live cells and represent the mean+S.E.M of two experiments done in duplicates. **p<0.01; ***p<0.001 vs. control; #p<0.05 with respect to COMP-4.

FIGS. 4A and 4B show the effect of COMP-4 on mRNA (messenger ribonucleic acid) and protein expression of nNOS (neuronal nitric oxide synthase), eNOS (endothelial nitric oxide synthase), and iNOS. HUAEC cells were incubated with COMP-4 for 24 hours. FIG. 4A shows mRNA expression for nNOS, eNOS, and iNOS was determined by qPCR (quantitative polymerase chain reaction). Results were expressed as a fold increase with respect to the control. **p<0.01 with respect to eNOS and iNOS control. FIG. 4B shows protein expression for nNOS, eNOS, and iNOS was determined by western blot. Results were expressed as ratio NOS/GAPDH (nitric oxide synthase/Glyceraldehyde 3-phosphate dehydrogenase) with respect to the control for each isoenzyme. *p<0.05 and ***p<0.001 with respect to control for eNOS and iNOS.

FIG. 5 shows the effect of COMP-4 on pro-inflammatory cytokine expression in HUAEC. Cytokine array analysis was performed using the cell culture media of HUAEC cells treated with or without COMP-4 for 24 hours. Only two cytokines out of forty in the array (IL-8 (interleukin-8) and PAI-1 (plasminogen activator inhibitor 1)) were expressed in the control group. DETA NONOate was used as NO donor. Results were expressed as normalized density regarding the reference spots and represent the mean+S.E.M. of two duplicated experiments. *p<0.05 and ***p<0.001 with respect to control.

FIG. 6 shows the effect of COMP-4 in mitigating H₂O₂-induced endothelial damage. Cells were treated with or without COMP-4 plus 100 μM H₂O₂ (hydrogen peroxide) for 24 hours. Co-incubation with COMP-4 and H₂O₂ increased nitrite formation. *p<0.05; **p<0.01 with respect to control; ###p<0.001 with respect to H₂O₂.

FIG. 7 shows the effect of COMP-4 on the expression of cytokines in H₂O₂-treated cells. Cytokine array analysis was performed using the cell culture media of HUAEC cells treated with or without COMP-4 and with the addition of H₂O₂ for 24 hours. Five out of forty cytokines in the array (MIF (Macrophage migration inhibitory factor), IL-8, IL-6 (interleukin-6), CXCL1 (chemokine ligand 1), and PAI-1) were expressed in the H₂O₂ group, and co-incubation with COMP-4 reduced the expression of these cytokines. Results are expressed as normalized density regarding the reference spots and represent the mean+S.E.M. of two duplicated experiments. *p<0.05; **p<0.01; ***p<0.001 with respect to control; ## p<0.01; ### p<0.001 with respect to H₂O₂.

FIGS. 8A and 8B show the effect of COMP-4 on PAI-1 expression and activity in H₂O₂-treated cells. FIG. 8A shows PAI-1 expression was determined by western blot in cell homogenate, and the supernatant of the HUAEC treated with H₂O₂ with or without COMP-4 for 24 hours. *p<0.05; **p<0.01 with respect to control; ## p<0.01; ###p<0.001 with respect to H₂O₂. FIG. 8B shows PAI-1 activity was quantified using an indirect colorimetric assay. H₂O₂-treated HUAEC showed increased PAI-1 activity, which was abrogated by COMP-4 treatment. *p<0.05 with respect to control; #p<0.05 versus H₂O₂ treatment alone.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

Impairment in nitric oxide (NO) production and/or activity is a hallmark of endothelial dysfunction, the putative clinical predictor of future cardiovascular events. A nutraceutical composition (which is referenced herein as COMP-4), and which was developed by Applicant, has been shown to stimulate the production of NO in a variety of tissues. As disclosed below in greater detail, the nutraceutical composition is composed of (1) ginger or ginger derivative, (2) L-citrulline, L-arginine, or mixture of L-arginine and L-citrulline and/or L-arginine, (3) muira puama, and (4) Paullinia cupana. It has now been determined the nutraceutical composition has a similar stimulatory effect on the arterial endothelial cell and as a result potentially guards against or prevents endothelial dysfunction.

In the body, NO is produced by a family of three enzymes called nitric oxide synthases (NOS) that use the amino acid, L-arginine, to produce NO. The isozyme of NOS within the endothelial cells that produces NO during its normal physiological processes is called endothelial NOS (eNOS). However, during times of stress involving the endothelial cells, NO can also be produced within the endothelial cells by another NOS isozyme to protect the cells against oxidative damage and cellular dysfunction. The NO that is induced in such a scenario is synthesized by the inducible NOS isozyme (iNOS) which is upregulated during times of stress to produce NO for its protective anti-oxidative effects.

It is also appreciated that the production of NO by the arterial endothelium, the inner lining of the blood vessel, is responsible for a) the ability of the blood vessel to dilate so it can increase its blood flow and b) act as an anti-clotting product to prevent blood clotting in those vessels. Under physiological stress either due to the development of a disease such as diabetes or simply from aging, the endothelial cells can be impacted and become dysfunctional thereby impairing their ability to make NO and even promote the development of blood clots. When such endothelial dysfunction occurs, it may be a precursor for the future development of cardiovascular (CV) disease like hypertension or even coronary artery disease. Therefore, the ability to somehow enhance the local production or availability of NO within such affected blood vessels in patients identified as prone to endothelial dysfunction could play a positive role in either preventing or delaying the onset of endothelial dysfunction and subsequent CV disease in such patients.

NO is not only an essential mediator in regulating endothelial cell function, but is also synthesized by the endothelial cell itself. NO regulates numerous biochemical activities within endothelial cells, such as maintenance of endothelial integrity, reduction of platelet deposition and aggregation, decrease in leukocyte adhesion, inhibition of smooth muscle cell proliferation and migration, and induction of vasodilation. Tousoulis D, Kampoli A M, Tentolouris C, Papageorgiou N, Stefanadis C. The role of nitric oxide on endothelial function. Curr Vasc Pharmacol. 2012 January; 10(1):4-18. doi: 0.2174/157016112798829760. PMID: 22112350; Ramamurthi A, Lewis R S. Nitric oxide inhibition of platelet deposition on biomaterials. Biomed Sci Instrum. 1999; 35:333-8. PMID: 11143374; Kubes P, Suzuki M, Granger D N. Nitric oxide: an endogenous modulator of leukocyte adhesion. Proc Natl Acad Sci U S A. 1991 Jun. 1; 88(11):4651-5. doi: 10.1073/pnas.88.11.4651. PMID: 1675786; PMCID: PMC51723; Zhang J, Zhao F, Yu X, Lu X, Zheng G. MicroRNA-155 modulates the proliferation of vascular smooth muscle cells by targeting endothelial nitric oxide synthase. Int J Mol Med. 2015 June; 35(6):1708-14. doi: 10.3892/ijmm.2015.2181. Epub 2015 Apr. 14. PMID: 25872580; Ignarro L J. Endothelium-derived nitric oxide: actions and properties. FASEB J. 1989 January; 3(1):31-6. doi: 10.1096/fasebj.3.1.2642868. PMID: 2642868; Lu X, Dang C Q, Guo X, Molloi Molloy S, Wassall C D, Kemple M D and Kassab G S: Elerated oxidative stress and endothelial dysfunction in the right coronary artery of right ventricular hypertrophy. J Appl Physiol (1985). 110:1674-1681. 2011. A reduced NO bioavailability at the endothelial cell layer contributes to endothelial dysfunction, which enhances the development of vascular fibrosis and artherosclerosis. Cyr A R, Huckaby L V, Shiva S S, Zuckerbraun B S. Nitric Oxide and Endothelial Dysfunction. Crit Care Clin. 2020 April; 36(2):307-321. doi: 10.1016/j.ccc.2019.12.009. PMID: 32172815; Lloyd-Jones D M, Bloch K D. The vascular biology of nitric oxide and its role in atherogenesis. Annu Rev Med 1996; 47:365-375; Kawashima S, Yokoyama M. Dysfunction of endothelial nitric oxide synthase and atherosclerosis. Arterioscler Thromb Vasc Biol. 2004 June; 24(6):998-1005. doi: 10.1161/01.ATV.0000125114.88079.96. Epub 2004 Mar. 4. PMID: 15001455.

It is assumed that the constitutively expressed eNOS isozyme is responsible for maintaining normal endothelial function. However, its function can be impacted by a variety of chronic inflammatory conditions that affect the vascular system, e.g., atherosclerosis. Kawashima S, Yokoyama M. Dysfunction of endothelial nitric oxide synthase and atherosclerosis. Arterioscler Thromb Vasc Biol. 2004 June; 24(6):998-1005. doi: 10.1161/01.ATV.0000125114.88079.96. Epub 2004 Mar. 4. PMID: 15001455; El Accaoui R N, Gould S T, Hajj G P, Chu Y, Davis M K, Kraft D C, Lund D D, Brooks R M, Doshi H, Zimmerman K A, Kutschke W, Anseth K S, Heistad D D, Weiss R M. Aortic valve sclerosis in mice deficient in endothelial nitric oxide synthase. Am J Physiol Heart Circ Physiol. 2014 May; 306(9): H1302-13.doi: PMID: 24610917; PMCID: PMC4010666. However, under chronic pro-inflammatory conditions, a local expression of the iNOS isotype can be seen in the endothelial cell as well as other cell types. MacNaul K L, Hutchinson N I. Differential expression of iNOS and cNOS mRNA in human rascular smooth muscle cells and endothelial cells under normal and inflammatory conditions. Biochem Biophys Res Commun. 1993 Nov. 15; 196(3):1330-4. doi: 10.1006/bbrc.1993.2398. PMID: 7504476. Activation of iNOS leads to high-output NO synthesis, which was initially perceived as a response associated with local tissue destruction and cell death. However, more recent investigations have demonstrated a powerful protective effect of NO synthesized from iNOS toward cellular stress conditions. Hemmrich K, Suschek C V, Lerzynski G, Kolb-Bachofen V. iNOS activity is essential for endothelial stress gene expression protecting against oxidative damage. J Appl Physiol (1985). 2003 November; 95(5): 1937-46. doi: 10.1152/japplphysiol.00419.2003. Epub 2003 Jul. 25. PMID: 12882997. Moreover, it has been shown that iNOS-derived NO modulates the expression of many different genes that promote protective responses during pro-inflammatory conditions and reduces the injury response and atherosclerotic development in arteries. Zhen J, Lu H, Wang X Q, Vaziri N D, Zhou X J. Upregulation of endothelial and inducible nitric oxide synthase expression by reactive oxygen species. Am J Hypertens. 2008 January; 21(1):28-34. doi: 10.1038/ajh.2007.14. PMID: 18091741; Raman K G, Gandley R E, Rohland J, Zenati M S, Tzeng E. Early hypercholesterolemia contributes to vasomotor dysfunction and injury associated atherogenesis that can be inhibited by nitric oxide. J Vasc Surg (2011) 53:754-63. doi:10.1016/j.jvs.2010.09.038

Oxidative stress is considered a crucial pathogenic factor for endothelial cell injury and death. The accumulation of reactive oxygen species, reactive nitrogen species, and cytokine activation is associated with many forms of vascular dysfunction. Clapp B R, Hingorani A D, Kharbanda R K, Mohamed-Ali V, Stephens J W, Vallance P, and MacAllister R J: Inflammation-induced endothelial dysfunction involves reduced nitric oxide bioavailability and increased oxidant stress. Cardiovasc Res. 64:172-178. 2004. One such cytokine marker is PAI-1, which is upregulated in activated or injured endothelial and vascular smooth muscle cells. Diebold I, Kraicun D, Bonello S, Görlach A. The ‘PAI-1 paradox’ in vascular remodeling. Thromb Haemost. 2008 December; 100(6):984-91. PMID: 19132221; Samarakoon R, Higgins P J. The Cytoskeletal Network Regulates Expression of the Profibrotic Genes PAI-1 and CTGF in Vascular Smooth Muscle Cells. Adv Pharmacol. 201 8; 81:79-94. doi: 10.1016/bs.apha.2017.08.006. PMID: 29310804. Increased expression of PAI-1 suppresses the fibrinolytic system and creates a prothrombotic state, resulting in the pathological deposition of fibrin, clot formation, and subsequent tissue damage. In vivo, this increased expression of PAI-1 by the endothelial and possibly the underlying vascular smooth muscle cells is associated with the development of vascular thrombosis, fibrosis, and subsequent cardiovascular disease. Diebold I, Kraicun D, Bonello S, Görlach A. The ‘PAI-1 paradox’ in vascular remodeling. Thromb Haemost. 2008 December; 100(6):984-91. PMID: 19132221; Samarakoon R, Higgins P J. The Cytoskeletal Network Regulates Expression of the Profibrotic Genes PAI-1 and CTGF in Vascular Smooth Muscle Cells. Adv Pharmacol. 201 8; 81:79-94. doi: 10.1016/bs.apha.2017.08.006. PMID: 29310804.

The present invention provides a nutraceutical composition, COMP-4, and associated method for the protection of human arterial endothelial cells from endothelial dysfunction, through the endogenous production of NO by the iNOS enzyme as well as stimulation of the constitutively expressed eNOS isozyme within the endothelial cell. As discussed below in more detail, this upregulation of the iNOS-NO-cGMP pathway together with stimulation of the eNOS-NO-cGMP pathway within the endothelial cells is proposed to interfere or block the levels of PAI-1 and the negative effects on the cardiovascular system known to be associated with the upregulation of PAI-1.

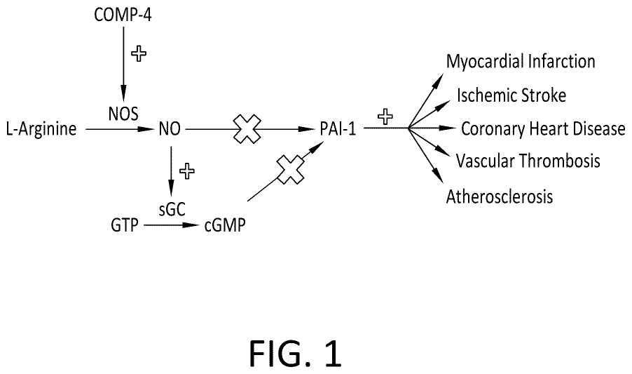

As shown with reference to FIG. 1, NO is known to be an important signaling molecule in many physiological systems. NO is also known as a potent antioxidant and anti-apoptotic molecule (meaning that it fights cellular death). NO itself can either work directly inside the mitochondria to quench or stop oxidative stress, or the NO, via stimulation of sGC (soluble guanylyl cyclase), can convert a molecule called GTP (Guanosine triphosphate) to cGMP which can act as a second messenger to affect the function of the endothelial cell. COMP-4 upregulates the iNOS-NO-cGMP pathway together with stimulation of the eNOS-NO-cGMP pathway within the tissues, and in so doing inhibits the production of PAI-1.

Applicant has determined that in order to upregulate the iNOS-NO-cGMP pathway together with stimulation of the eNOS-NO-cGMP pathway, and ultimately inhibit the production of PAI-1, it is necessary to increase NOS enzyme production and stimulate sGC (soluble guanylyl cyclase) to make cGMP from GTP. COMP-4 achieves these goals. The results of the testing presented below demonstrate that in arterial endothelial cells, COMP-4 activates both the iNOS-NO-cGMP and eNOS-NO-cGMP pathways to increase the production and bioavailability of NO and thus protect the arterial endothelial cells and blood vessel from the effects of endothelial dysfunction.

The nutraceutical composition, COMP-4, has been shown previously by Applicant to stimulate the production of NO from iNOS in a variety of cell types. Applicant has previously demonstrated that COMP-4—a compound consisting of ginger, L-citrulline, and the herbal components Paullinia cupana and muira puama—reverted the apoptosis, fibrosis, and oxidative stress observed in aging rats' cavernosal smooth muscle tissue, resulting in an improvement in the aged animals' erectile function. Ferrini M G, Hlaing S M, Chan A, et al. Treatment with a combination of ginger, L-citrulline, muira puama and Paullinia cupana can reverse the progression of corporal smooth muscle loss, fibrosis and veno-occlusive dysfunction in the aging rat. Andrology (Los Angel). 2015 June; 4(1):132. Doi: 10.4172/2167-0250.1000132. Epub 2015 May 25. PMID: 26405615; PMCID: PMC4578663. It was further determined that COMP-4 induced these histological and physiological effects through activation and upregulation of NO and cGMP from the normally dormant iNOS-NO-cGMP pathway within the rat cavernosal smooth muscle cells. Ferrini MG, Garcia E, Abraham A, Artaza J N, Nguyen S, Rajfer J. Effect of ginger, Paullinia cupana, muira puama and l-citrulline, singly or in combination, on modulation of the inducible nitric oxide-NO-cGMP pathway in rat penile smooth muscle cells. Nitric Oxide. 2018 Jun. 1; 76:81-86. Doi: 10.1016/j.niox.2018.03.010. Epub 2018 Mar. 16. PMID: 29551532. COMP-4 was subsequently shown to have the same effect on the iNOS-NO-cGMP pathway in human cavernosal smooth muscle cells in vitro. Ferrini M G, Abraham A, Graciano L, Nguyen S, Mills J N, Rajfer J. Activation of the iNOS/NO/cGMP pathway by Reractin® in human corporal smooth muscle cells. Transl Androl Urol. 2021 July; 10(7):2889-2898. Doi: 10.21037/tau-21-11. PMID: 34430391; PMCID: PMC8350259. In addition, COMP-4 has been shown in osteoblast cells to stimulate NO not only from iNOS induction but also by upregulating eNOS, leading to accelerated fracture healing and prevention of osteoporosis in ovariectomized rats. Rajfer R A, Kilic A, Neviaser A S, Schulte L M, Hlaing S M, Landeros J, Ferrini M G, Ebramzadeh E, Park S H. Enhancement of fracture healing in the rat, modulated by compounds that stimulate inducible nitric oxide synthase: Acceleration of fracture healing via inducible nitric oxide synthase. Bone Joint Res. 2017 Febuary; 6(2):90-97. Doi: 10.1302/2046-3758.62.BJR-2016-0164.R2. PubMed PMID: 28188129; PubMed Central PMCID: PMC5331177; Rajfer R. A., Flores M, Abraham A, Garcia E., Hinojosa N., Desai M., Artaza J. N., Ferrini M. G. Prevention of Osteoporosis in the Orariectomized Rat by Oral Administration of a Nutraceutical Combination That Stimulates Nitric Oxide Production. J Osteoporos. 2019 Jun. 2; 2019:1592328. Doi: 10.1155/2019/1592328. PMID: 31275540; PMCID: PMC6582785.

Given the increasing evidence that COMP-4 stimulates the iNOS-NO-cGMP pathway in both rat and human cavernosal smooth muscle cells, along with its action on eNOS in osteoblasts, Applicant has determined the compound has a similar effect on the vascular arterial endothelial cell and thereby affects endothelial function in general. To this end, Applicant conducted an in vitro study (presented below) to evaluate the impact of COMP-4 in a human arterial endothelial cell line with or without endothelial dysfunction, with a focus on cGMP expression, NO activity, NOS expression, and PAI-1 activity.

As briefly discussed below, COMP-4 comprises effective amounts of ginger or a ginger derivative selected from the group consisting of fresh, partially dried vegetable ginger, dried vegetable ginger, 6-gingerol and mixtures thereof; at least one of the group consisting of arginine and citrulline (preferably, L-arginine, L-citrulline, or a combination of L-arginine and L-citrulline); muira puama; and Paullinia cupana (guarana).

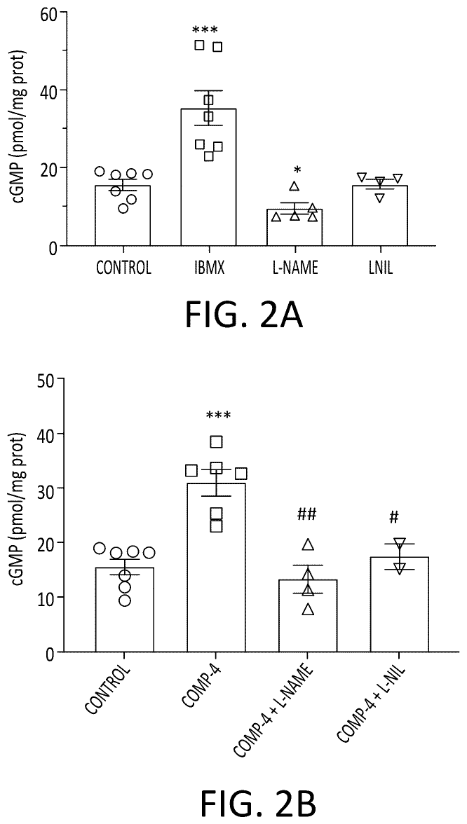

In many of the tissues within the human body, it is well known that NO per se is one of the main signaling molecules that the body uses to organize how the majority of cells pursue their normal physiological roles. The NO involved with these functions is synthesized from one of the three isoforms of NOS. The eNOS isoform as well as the neuronal isoforms of NOS (nNOS) are constitutively expressed while the iNOS isoform is induced by some stimulant. Applicant's prior work shows COMP-4 is known to stimulate iNOS in the cavernosal smooth muscle cells (see U.S. Patent Application Publication No. 2014/0255528, entitled “COMPOSITIONS AND METHODS FOR TREATING, INHIBITING THE ONSET, AND SLOWING THE PROGRESSION OF ERECTILE DYSFUNCTION INCLUDING NATURALLY OCCURRING AGE RELATED ERECTILE DYSFUNCTION”, which is incorporated herein by reference) as well as in the osteoblast cells (see U.S. Patent Application Publication No. 2018/0104300, entitled “COMPOSITIONS AND METHODS FOR THE TREATMENT OF ORTHOPEDIC AILMENTS”, which is incorporated herein by reference). As the nutraceutical composition of the present invention has been shown to stimulate iNOS, it is conceived that the nutraceutical composition of the present invention stimulates the iNOS-NO-cGMP pathway in a manner having a positive effect on the regulation of endothelial cell function such as inhibition of the production of PAI-1. A study, the results of which are shown with reference to FIGS. 2 to 8, was conducted to study the impact of COMP-4 in the HUAEC in the presence of absence of H₂O₂-induced endothelial dysfunction.

Considering the individual components of the nutraceutical composition in accordance with the present invention, ginger is a complex natural composition having numerous purported properties when used alone and/or in combination with other compounds. For example, some traditional Chinese medicines have used or included ginger in compositions to treat or prevent various maladies based on a variety of metaphysical reasons. However, over the past century, scientific methods have shown that many traditional Chinese medicines do not produce the purported effects and/or may even make the target maladies worse. Nevertheless, some traditional Chinese medicines have been found to contain active agents that may be of medical use, even if not effective or safe for the use purported by traditional Chinese medicine. The complexity of ginger and its myriad properties is reflected by certain constituent compounds which have the following structure:

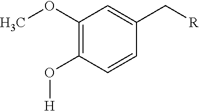

wherein, for example, in 6-gingerol the R sidechain of the vanillyl function group (i.e., 4-hydroxy-3-methoxyphenyl group) is:

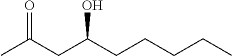

Thus, 6-gingerol (also called gingerol) is (S)-5-hydroxy-1-(4-hydroxy-3-methoxyphenyl)-3-decanone and has the following structure:

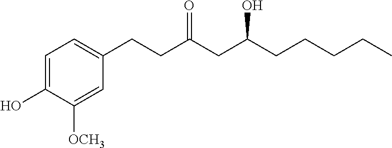 [SMILES: CCCCC[C@H](O)CC(=O)CCc1ccc(O)c(OC)c1]

Since ginger contains multiple compounds, of varying complexity and chemical activity, there are conflicting teachings in the art about the biological activity of compounds that might be useful in inducing NO production or otherwise having a potential role in treatment. Further, there remains considerable unpredictability about how to understand, much less control, the relevant metabolic pathways.

Ginger, when combined with muira puama, Paullinia cupana (guarana), and at least one of the group consisting of arginine and citrulline (preferably, L-arginine, L-citrulline, or a combination of L-arginine and L-citrulline) is effective in promoting the production of NO directly by the cell in amounts greater than each of the four individual components by themselves resulting in significantly high levels of NO.

An oral dosage form of the nutraceutical composition in accordance with the present invention is selected from the group consisting of a tablet, capsule, lozenge, powder, or suspension comprising the foregoing ingredients. Preferred suspensions are aqueous and/or alcohol (ethanol) based.

The raw materials and ingredient matter may be dried, for example by freeze-drying or vacuum drying, before compounding into oral dosage forms. Individual dosage forms may comprise compressed tablets, capsules, lozenges or may be provided in sachets. Suspension formulations may be provided. Ginger, ginger root extract, L-arginine, L-citrulline, muira puama and Paullinia cupana are all separately commercially available, with preferred sources and analyses provided infra. Preferably, the ingredients are combined and encapsulated in gelatin capsules, but other dosage forms are anticipated that will produce equivalent results.

Flavorings or taste masking agents may be employed. Tablets or other dosage forms may include diluents (e.g., lactose), disintegrants (e.g., cross carmelose sodium), or binders (e.g., polyvinylpyrollidone). Lubricants for example magnesium stearate, or other conventional excipients may be employed (e.g., silicas, carbohydrates, etc.). Film-coated tablets may be provided.

The active ingredients of composition of the present invention are combined using well known and standard processes and agents. Preferably, a gelatin capsule contains the combined ingredients in powder form. Standard ingredients in powder formulations are used for preparing and compounding preferred exemplary formulations of the present inventions. The ingredients, in powder form, are inspected, weighed, blended, and encapsulated in gelatin capsules. The blending process includes standard screening, blending and metal detection at standard temperatures and in a sterile environment at least sufficient for food supplements.

Sources of active ingredients may include:

### Exemplary Active Ingredient Sources

### Ginger

SUPPLIER 1. SOLARAY GINGER Root Extract
Ginger root—250 mg (5% gingerols)

Gingerols—12.5 mg/5%
Other ingredients: Magnesium Stearate, Croscamellose Sodium
Park City, UT
www.Solaray.com
SUPPLIER 2. NATURE'S ANSWER, INC. Ginger Rhizome Extract
Ginger Rhizome Extract—125 mg standardized for 5% gingerols+shogoals)
Other ingredients: Vegetable Cellulose, Rice Flour, Di-Calcium Phosphate, Calcium Silicate
Hauppauge, NY 11788-3943
http://www.naturesanswer.com/
SUPPLIER 3. SOLGAR GINGER Root Extract
Ginger Root Extract—300 mg (5% ginger phenols)
Raw Ginger Powder—150 mg
C-ascorbic acid, beta-carotene, magnesium stearate, monocrystalline cellulose
Veronica, NJ
SOLGAR GINGER Powder
Ginger powder—500 mg
Root Ginger Extract 4:1-5 mg

### L-Citrulline

SOURCE NATURALS L-Citrulline Free-Form Amino Acid Supplement:
L-Citrulline 2 g
Other Ingredients: gelatin (capsule), microcrystalline cellulose, colloidal silicon dioxide, and magnesium stearate.
Source Naturals, Inc.
P.O. Box 2118
Santa Cruz, CA 95062
http://www.sourcenaturals.com/

### L-Arginine

THE VITAMIN SHOPPE L-ARGININE
L-Arginine—500 mg
Vitamin B6 10 mg
Other Ingredients: gelatin, rice flour, magnesium stearate

### Muira Puama

SUPPLIER 1. SOLARAY Muira puama Ptychopetalum Olacoides Dietary
Supplement:
Muira puama Ptychopetalum Olacoides (root)—600 mg
Other Ingredients: Gelatin Capsule, and Cellulose.
Manufactured by Nutraceutical Corp.
Park City, UT 84060
http://www.solaray.com
SUPPLIER 2. NATURE'S ANSWER Muira puama Organic Alcohol Extract:
Muira puama Root Extract (1:1)—2000 mg
Other Ingredients: Purified Water, Vegetable Glycerin, 12-15% Certified
Organic Alcohol

### Paullinia Cupana (Guarana)

SOURCE NATURALS Guarana Energizer Dietary Supplement:
Guarana Seed Extract (22% caffeine)—900 mg
Other Ingredients: Microcrystalline cellulose, dibasic calcium phosphate, stearic acid, modified cellulose gum, and colloidal silicon dioxide.
Source Naturals, Inc.
P.O. Box 2118
Santa Cruz, CA 95062
http://www.sourcenaturals.com/

“Effective amount” as used in the present disclosure is intended to mean that a dosage form of the nutraceutical composition contains an amount of each ingredient sufficient when administered to a human patient for a sufficient period of time to produce a beneficial effect, that is, a statistically significant reduction in PAI-1.

“Individual Dosage” as used herein means the amount of the nutraceutical composition in a single dose of the nutraceutical composition administered to a human patient as part of a dosing regimen.

“Total Daily Dosage” as used herein means the cumulative amount of the nutraceutical composition administered to a human patient over the course of a day whether the nutraceutical composition is administered once a day or multiple times a day as part of a dosing regimen.

“About” as used herein refers to a range of values +/−10% of a specified value.

In accordance with a preferred embodiment, the total daily dosage of the nutraceutical composition is
up to about 3 g (preferably about 250 mg to about 2 g) ginger or ginger derivative,
about 10 mg to about 2 g (preferably about 400 mg to about 2 g) of a L-citrulline, L-arginine or a combination of L-arginine and L-citrulline,
about 100 mg to about 3 g (preferably about 500 mg to about 1.5 g) Muira puama, and
at least about 125 mg (preferably about 500 mg) Paullinia cupana (guarana).

A specific individual dosage forming the basis for the test results presented herein, where the nutraceutical composition is taken only once a day, includes
about 500 mg ginger or ginger derivative,
about 1,600 mg L-citrulline,
about 500 mg Muira puama, and
about 500 mg Paullinia cupana.
   While a daily administration of the nutraceutical composition of the present invention is preferred, it is appreciated the nutraceutical composition may be administered multiple times a day and the individual dosages would therefore be adjusted so as to not exceed the preferred total daily dosage as outlined above. Whether the nutraceutical composition is administered once a day or multiple times throughout the day, the nutraceutical composition is administered for a sufficient period of time.

The nutraceutical composition may include optional pharmaceutically acceptable excipients, fillers, binders, and colorants, and can be packaged in standard gelatin capsules or formed into solid tablets, taken in particulate form, or mixed into and/or suspended in solution.

As briefly mentioned above, Applicant performed studies regarding the efficacy of COMP-4. As will be appreciated based upon the following results, the study was performed using HUAEC. The HUAEC were incubated for 24 hours with or without COMP-4 and, in order to replicate endothelial dysfunction, co-incubated with or without H₂O₂. NO intracellular content was determined by flow cytometry and DAF-FM DA staining. In cell culture media, nitrite formation was measured by a colorimetric assay, while cGMP content was determined by ELISA. The three NOS isoforms and their mRNA content were measured by Western blot (WB) and RT-PCR, respectively. Expression of pro-inflammatory cytokines was determined by a cytokine array. PAI-1 expression and activity were measured by WB and an indirect colorimetric activity assay. An NO donor was used as a positive control, and L-NAME and L-NIL were used as non-selective and iNOS-selective NOS inhibitors, respectively, for negative control.

The results show that COMP-4 increased the endothelial cell production of NO and cGMP as well as the expression of both eNOS and iNOS, in tandem with a reduction in cytokine expression and activity of PAI-1. The co-incubation of COMP-4 with H₂O₂ reversed the detrimental effects of H₂O₂ on endothelial function as evidenced by improvement in NO availability and abrogation of the pro-inflammatory milieu.

Based upon the results of Applicant's study, it is found that COMP-4 leads to an increase in the production of NO as a result of its stimulatory effect on both endothelial-derived eNOS and iNOS. This increase in NO bioavailability within the endothelial cell by COMP-4 leads to a reduction in pro-inflammatory cytokines, in particular the prothrombotic PAI-1, thereby suggesting a role for COMP-4 in either the treatment or prevention of endothelial dysfunction.

The study was performed in the following manner:

### 1. MATERIALS AND METHODS

### 1.1 Cell Culture

Frozen human umbilical arterial endothelial cells (HUAEC, cat #202-05n, Cell Applications, Inc., San Diego, CA) were thawed in a 37° C. water bath. Cells were resuspended and dispensed into a coated T75 flask. Cells were seeded at a density of 5,000-7,000 cells/cm2 in Human Meso Endo Growth Medium (cat #212-500, Cell Applications), left undisturbed for 16 hours, and refreshed the following day with supplemented culture medium. Medium was changed every three days until the culture reached 90% confluency. Cells were then split and sub-cultured for conducting the experiments at a density of 3.8 x 105 cells per well at 100% confluency.

### 1.2 Reagents

COMP-4 was produced by combining muira puama (MP: 0.9 mg/ml), Paullinia cupana (PC: 0.9 mg/ml), ginger (G: 0.225 mg/ml), and L-citrulline (0.9 mg/ml). The muira puama, Paullinia cupana, and ginger were donated by Naturex, South Hackensack, NJ. L-citrulline was obtained from Sigma-Aldrich, St. Louis, MO.

For COMP-4, all four individual components were mixed, reaching a final concentration of 0.69 mg/ml. Paullinia cupana, muira puama, and L-citrulline were prepared at a 100-fold stock solution. Due to its solubility, ginger was prepared in a 200-fold stock solution, as previously described. Ferrini M G, Garcia E, Abraham A, Artaza J N, Nguyen S, Rajfer J. Effect of ginger, Paullinia cupana, muira puama and L-citrulline, singly or in combination, on modulation of the inducible nitric oxide-NO-cGMP pathway in rat penile smooth muscle cells. Nitric Oxide. 2018 Jun. 1; 76:81-86. doi: 10.1016/j.niox.2018.03.010. Epub 2018 Mar. 16. PMID: 29551532. All the components were dissolved in 70% ethanol, except for L-citrulline, which was dissolved in water. 10 μL of the COMP-4 mixture were added per mL of media. In all the experiments performed, control cells received the same amount of ethanol as treated cells. Ferrini M G, Garcia E, Abraham A, Artaza J N, Nguyen S, Rajfer J. Effect of ginger, Paullinia cupana, muira puama and L-citrulline, singly or in combination, on modulation of the inducible nitric oxide-NO-cGMP pathway in rat penile smooth muscle cells. Nitric Oxide. 2018 Jun. 1; 76:81-86. doi: 10.1016/j.niox.2018.03.010. Epub 2018 Mar. 16. PMID: 29551532.

In addition, several inhibitors of the NO-cGMP pathway were employed in the experiments. L-NIL, a specific inhibitor of iNOS, was used at 2 μM; L-NAME, a non-selective inhibitor of all three NOS isoforms, was used at 3 μM. IBMX, an inhibitor of the phosphodiesterase enzyme, was used at 0.4 mM.

It has been shown that H₂O₂ is a crucial member of the class of reactive oxygen species (ROS), which is generated via the respiratory chain cascade and is a byproduct of cellular metabolism. H₂O₂ can also penetrate the plasma membrane and cause injury and apoptosis of endothelial cells, and as such has been extensively used in in vitro models to induce endothelial dysfunction. Lennicke, C., Rahn, J., Lichtenfels, R. et al. Hydrogen peroxide-production, fate and role in redox signaling of tumor cells. Cell Commun Signal 13, 39 (2015). https://doi.org/10.1186/s12964-015-0118 6; Han, R., Tang, F., Lu, M., Xu, C., Hu, J., Mei, M., & Wang, H. (2017). Astragalus polysaccharide ameliorates H₂O₂-induced human umbilical vein endothelial cell injury. Molecular Medicine Reports, 15, 4027-4034; Zheng Z, Wang M, Cheng C, Liu D, Wu L, Zhu J, Qian X. Ginsenoside Rb1 reduces H₂O₂ induced HUVEC dysfunction by stimulating the sirtuin 1/AMP activated protein kinase pathway. Mol Med Rep. 2020 July; 22(1):247-256. doi: 10.3892/mmr.2020.11096. Epub 2020 Apr. 28. PMID: 32377712; PMCID: PMC7248484. Therefore, to induce a state resembling endothelial dysfunction, a series of experiments were conducted using H₂O₂ at a concentration of 100 μM. Zheng Z, Wang M, Cheng C, Liu D, Wu L, Zhu J, Qian X. Ginsenoside Rb1 reduces H₂O₂ induced HUVEC dysfunction by stimulating the sirtuin 1/AMP activated protein kinase pathway. Mol Med Rep. 2020 July; 22(1):247-256. doi: 10.3892/mmr.2020.11096. Epub 2020 Apr. 28. PMID: 32377712; PMCID: PMC7248484. All incubations with the vehicle, COMP-4, inhibitors, and H₂O₂ were conducted for 24 hours.

### 1.3 Determination of cGMP Expression

HUAEC were seeded at 3.8×105 cells per well in a 6-well plate, and after reaching confluency, cells were incubated with vehicle, COMP-4, IBMX, L-NIL, or L-NAME for 24 hours.

After incubation, the media were removed, and a 400 μL HCl 0.1 M was added for 20 minutes. Cells were then scraped, homogenized by pipetting, and centrifuged at 1,000 g for 10 minutes. The supernatants were used to determine cGMP concentration by a colorimetric ELISA (cat #581021, Cayman Chemical Company, Ann Arbor, MI), following the manufacturer's instructions. The enzymatic reaction product was determined by spectrophotometry at 405 nm absorbance and expressed as pmol/mg protein.

### 1.4 Determination of Nitrite Formation

After incubation, the cell culture media were collected and frozen at −80° C. prior to determination of total nitrite concentration by the Griess reaction (cat #780001, Cayman Chemical Company). Griess reagents R1 followed by R2 were added to the nitrite blanks, nitrite standards, and each sample on a 96-well plate. After 10 minutes of incubation at room temperature, the absorbance was measured with a plate reader at 540 nm, and the nitrite concentration was measured as previously described. Ferrini M G, Abraham A, Graciano L, Nguyen S, Mills J N, Rajfer J. Activation of the iNOS/NO/cGMP pathway by ReractinR in human corporal smooth muscle cells. Transl Androl Urol. 2021 July; 10(7):2889-2898. doi: 10.21037/tau-21-11. PMID: 34430391; PMCID: PMC8350259; Ferrini M G, Abraham A, Nguyen S, Luna R, Flores M, Artaza J N, Graciano L, Rajfer J. Exogenous L-Arginine does not stimulate production of NO or cGMP within the rat corporal smooth muscle cells in culture. Nitric Oxide. 2019 Aug. 1; 89: 64-70. doi: 10.1016/j.niox.2019.05.004. Epub 2019 May 7. PMID: 31075315; PMCID: PMC6786268.

### 1.5 Detection of Intracellular Nitric Oxide Formation by Flow Cytometry and by Confocal Microscopy

Intracellular NO production was determined by flow cytometry and confocal microscopy using the NO-specific probe DAF-FM DA, a cell-permeable fluorescent probe for the detection of NO.

For confocal microscopy, HUAEC were seeded in 4-well chambers (Nunc™ Lab-Tek™, Thermo Fisher) at 40,000 cells per well and treated with or without COMP-4 for 4 and 24 hours. After incubation, the media was removed, and the cells were incubated with 5 μM DAF-FM DA (cat #D-23844, Thermo Fisher) in the dark for 40 min at 37° C. After washing with PBS 3 times, the counterstain DAPI was added to the chamber slides and incubated for 5 minutes. Fluorescence staining from the DAF-FM DA in the cells was detected using a Leica SPE confocal microscope. Pictures were taken at 40× magnification, and the merged images of DAF-FM DA and DAPI were obtained with the Leica Application Suite X (LAS X) photo documentation system. These experiments were repeated twice.

For flow cytometry, HUAEC seeded at 1.0×106 cells/ml in T25 flasks were treated with or without COMP-4 0.1M of the NO donor DETA NONOate (cat #82120, Cayman Chemical), and 5 μg/mL lipopolysaccharide (LPS, cat #00-4976-93, Invitrogen eBioscience.) for 24 hours. Cells were then incubated for 2 hours with 5 μM DAF-FM DA. Following incubation and washing, cells were treated with trypsin-EDTA (ethylenediaminetetraacetic acid) to detach gently before immediate flow cytometry analysis. During this part of the experiment, exposure to light was avoided due to the light sensitivity of the probe. The intracellular fluorescence of DAF-FM DA was analyzed by flow cytometry using Attune NxT (Thermo Fisher; excitation wavelength 488 nm, emission wavelength 519 nm). The results were expressed as percent DAF-FM DA in live cells.

### 1.6 Intracellular Nitric Oxide Activity

To corroborate the results obtained by flow cytometry, another system for detecting intracellular NO was used, the Muse® Nitric Oxide Kit (cat #MCH100112, Luminex, Austin, TX). HUAEC were seeded at 1.0×106 cells/ml in T25 flasks, treated with vehicle, COMP-4 with or without L-NAME, or LPS (positive control), and incubated for 24 hours. Cells were then suspended in assay buffer at 106 cells/ml. 100 μL of working solution consisting of the Muse Nitric Oxide Reagent (DAX-J2 Orange) was added to 10 μL of cells in suspension. Samples were incubated for 30 minutes in a 37° C. incubator with 5% CO₂. After the incubation, 90 μL of the Muse 7-AAD working solution (dead cell marker) was added to each tube and incubated at room temperature for 5 min, protected from light. Intracellular NO was measured using the Guava® Muse Cell Analyzer, calibrated with negative and positive controls. Generated plots were divided into quadrants of nitric oxide positivity (+or −) and dead cells (+or −). From each plot, the lower-right quadrant of nitric oxide (+) and dead cell marker (−) was used to express intracellular NO results as the percent positive NO in live cells, which was compared between different treatments.

### 1.7 Western Blotting and Densitometry Analysis

After incubating with vehicle and treatments, 30 μg of protein obtained from the cell lysates were subjected to gel electrophoresis with 4-15% Tris-HCl PAGE (Bio-Rad, Hercules, CA) in running buffer (Tris/glycine/SDS). The proteins were transferred onto polyvinylidene fluoride (PVDF) membranes embedded in transfer buffer (Tris/glycine/methanol) using transblot semi-dry apparatus (Bio-Rad, Hercules CA). The nonspecific binding was blocked by immersing the membranes into 5% nonfat dried milk and 0.1% (v/v) Tween 20 in PBS for 1 hour at room temperature, as previously described. Tousoulis D, Kampoli A M, Tentolouris C, Papageorgiou N, Stefanadis C. The role of nitric oxide on endothelial function. Curr Vasc Pharmacol. 2012 January; 10(1):4-18. doi: 0.2174/157016112798829760. PMID: 22112350; Samarakoon R, Higgins P J. The Cytoskeletal Network Regulates Expression of the Profibrotic Genes PAI-1 and CTGF in Vascular Smooth Muscle Cells. Adv Pharmacol. 201 8; 81:79-94. doi: 10.1016/bs.apha.2017.08.006. PMID: 29310804. After several washes with the washing buffer (PBS Tween 0.1%), the membranes were incubated with the primary antibodies overnight at 4° C. Primary mouse monoclonal and rabbit polyclonal antibodies were used for: iNOS at 1:250 dilution (Abcam cat #ab15323, RRID:AB_301857, Cambridge, UK), eNOS at 1:500 dilution (BD Biosciences cat #610299, RRID:AB_397693, San Jose, CA); nNOS at 1:500 dilution (Abcam cat #ab76067, RRID:AB_2152469), and PAI-1 at 1:2000 dilution (Abcam cat #ab182973). The membranes were incubated for 2 hours at room temperature with 1:2000 dilution of anti-mouse or anti-rabbit secondary antibody linked with HRP (Cell Signaling Technology, cat #7076, RRID:AB_330924 and cat #7074, RRID:AB_2099233, Danvers, MA). After several washes, the bands were visualized using the WesternSure PREMIUM chemiluminescent detection system (Li-COR Biotechnology cat #926-95000, Lincoln, NE). The bands of the respective analyte and the total protein content in each well were scanned by LI-COR Odyssey Fc Infrared Imaging System (LI-COR) and semi-quantified using ImageStudio (v5.2, LI-COR). The results were expressed as analyte/total protein normalized with respect to the control.

### 1.8 Quantitative Real-Time RT-PCR to Evaluate Expression of NOS Isotypes

RNA (ribonucleic acid) was extracted using Trizol Reagent (Invitrogen, Carlsbad, CA), and equal amounts (1 μg) of RNA were reverse transcribed using a high-capacity RNA-to-cDNA PCR kit (Applied Biosystems, Foster City, CA). The PCR primer set (RT2) for human iNOS, human nNOS, and human eNOS were obtained from SABiosciences (Germantown, MD). Real-time PCR (SYBR GreenER, Applied Biosystems) was performed using a StepOne Plus instrument (Applied Biosystems). The protocol included melting for 15 min at 95° C., 40 cycles of three-step PCR including melting for 15 s at 95° ° C., annealing for 30 s at 58° C., elongation for 30 s at 72° C. with an additional detection step of 15 s at 81° C., followed by a melting curve from 55 to 95° C. at the rate of 0.5° C.per 10 s. Experimental mRNA starting quantities were then calculated from the standard curves and averaged using SABiosciences software as previously described. Ferrini M G, Abraham A, Graciano L, Nguyen S, Mills J N, Rajfer J. Activation of the iNOS/NO/cGMP pathway by Reractin® in human corporal smooth muscle cells. Transl Androl Urol. 2021 July; 10(7):2889-2898. doi: 10.21037/tau-21-11. PMID: 34430391; PMCID: PMC8350259. The ratios of the experimental marker gene (e.g., iNOS, eNOS, or nNOS mRNA) to housekeeping gene RPLP1/3 mRNA were computed and normalized with control samples; Ferrini M G, Abraham A, Nguyen S, Luna R, Flores M, Artaza J N, Graciano L, Rajfer J. Exogenous L-Arginine does not stimulate production of NO or cGMP within the rat corporal smooth muscle cells in culture. Nitric Oxide. 2019 Aug. 1; 89: 64-70. doi: 10.1016/j.niox.2019.05.004. Epub 2019 May 7. PMID: 31075315; PMCID: PMC6786268.

### 1.9 Cytokine Array Expression

A cytokine/inflammation antibody array (R&D Systems, Minneapolis, MN) was used to assess the modulation of cytokines in HUAEC by COMP-4 and H₂O₂. The array was performed by following the manufacturer's instructions. Briefly, HUAEC were cultured on 6-well plates and treated for 24 hours with vehicle, COMP-4, the NO donor DETA NONOate, LPS (positive control of cytokine stimulation), H₂O_{2,} or the combination H₂O₂+COMP-4, prior to overnight serum starvation. The supernatant (500 μL.) was collected and incubated overnight with pre-blocked membranes spotted in duplicates with 36 antibodies for a variety of cytokines, chemokines, and acute-phase protein. An IR Dye 800CW Streptavidin (LI-COR, Catalog #926-32230) at 1:2000 dilution in array buffer was added to the membranes for 30 minutes at room temperature on a rocking platform. After several washes, the excess buffer was removed from the membranes by blotting the lower edge onto absorbent paper. The blotting images were scanned by LI-COR Odyssey FC Infrared Imaging System (LI-COR, Lincoln, NE), and cytokines were semi-quantified using ImageStudio (v5.2, LI-COR).

### 1.10 PAI-1 Activity

To assess the influence of COMP-4 on PAI-1 activity, HUAEC were seeded at equal densities in each well and grown on 6-well cell culture plates until they reached confluence. The monolayer of cells was incubated with vehicle or COMP-4 with or without H₂O₂ for 24 hours. The cell culture medium was collected and centrifuged at 3,000 g for 15 min and 4° C. to remove debris. The PAI-1 Activity Assay Kit from AbCam (cat #283368) was employed following the manufacturer's instructions. In brief, this is a two-step colorimetric assay for PAI-1. Samples are first incubated with a known amount of tissue plasminogen activator (tPA), allowing PAI-1 and tPA to form an inactive complex. Residual free tPA in the sample is exposed to a reaction mixture containing plasminogen and a chromogenic substrate cleaved by plasmin. Plasminogen is converted by the free tPA in the sample to plasmin, which acts on the substrate to release p-nitroaniline (p-NA). The absorbance of the released p-NA is inversely proportional to PAI-1 activity in the samples. Samples were assayed in duplicate.

### 1.11 Statistical Analysis

All data are presented as mean+S.E.M. Differences between groups were analyzed by one-way ANOVA followed by Dunnett's multiple comparisons tests using GraphPad Prism version 9.0.0 (GraphPad Software, San Diego, CA). All comparisons were two-tailed, and p<0.05 was considered statistically significant. Using simple randomization, cells were seeded in 6-well plates at 3.8×105 cells per well or 4-well chambers at 40,000 cells per well. All in vitro experiments were repeated three times, and data from representative experiments are shown.

### 2. RESULTS

### 2.1 COMP-4 Upregulates cGMP and NO Production in HUAEC

To determine whether the NO-cGMP pathway is functional in this cell line and whether COMP-4 has a regulatory effect on it, the expression of cGMP was studied. FIG. 2A shows that there was endogenous production of cGMP in this cell line, along with an increase in cGMP expression after treatment with the PDE inhibitor IBMX. When the cell line was incubated with the non-selective NOS inhibitor L-NAME, cGMP expression was reduced by 40%, whereas when incubated with the selective iNOS inhibitor L-NIL, there was no inhibition of cGMP expression.

FIG. 2B shows that incubation of the HUAEC with COMP-4 alone increased cGMP expression by 2-fold with respect to control, while co-incubation of COMP-4 with either L-NAME or L-NIL significantly reduced the cGMP expression by 57% and 44%, respectively, compared to COMP-4 incubation alone.

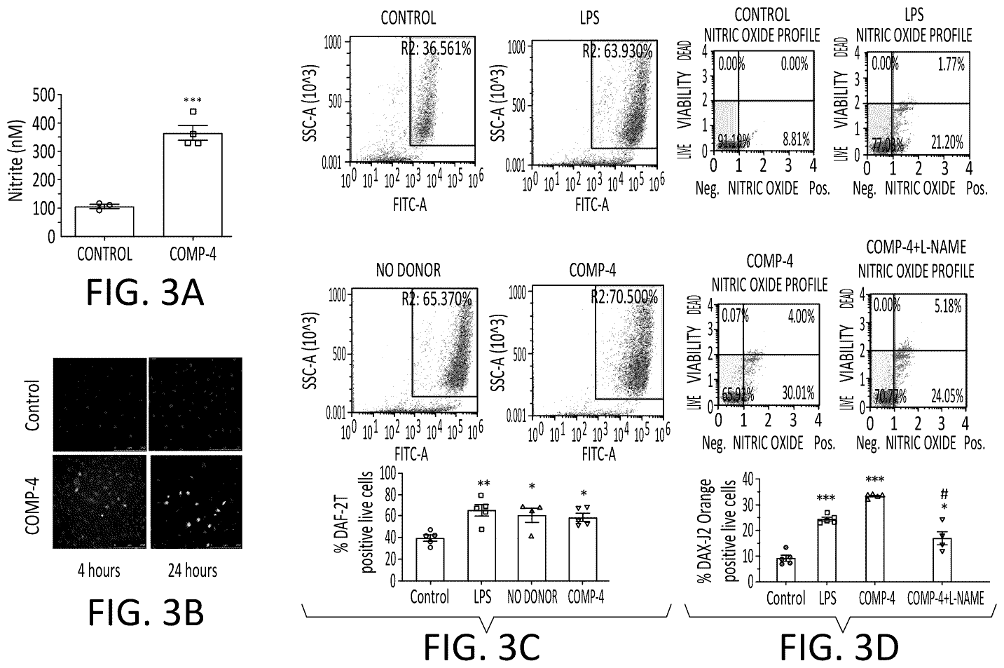

The production of NO by these same incubations was measured a) by nitrite formation in the cell culture media, b) intracellularly by DAF DA staining and flow cytometry, and c) by the use of the Luminex MUSE Nitric Oxide Kit. FIG. 3A shows that COMP-4 markedly increased nitrite formation in cell culture media compared to control (p<0).001). Because these absolute nitrite levels in the media of these human endothelial cells were much lower than those observed previously in human cavernosal smooth muscle cells (Ferrini M G, Abraham A, Graciano L, Nguyen S, Mills J N, Rajfer J. Activation of the iNOS/NO/cGMP pathway by Reractin® in human corporal smooth muscle cells. Transl Androl Urol. 2021 July; 10(7):2889-2898. doi: 10.21037/tau-21-11. PMID: 34430391; PMCID: PMC8350259), Applicant also studied the expression of NO intracellularly by three different methods. FIG. 3B shows the expression of DAF-2T (4,5-Diaminofluorescein triazole) staining, the product of the oxidation of DAF DA with NO, in the HUAEC cells with or without COMP-4 treatment at two time points. Positive staining of DAF-2T (white dots) was observed with COMP-4 treatment at 4 and 24 hours, whereas untreated controls showed faint expression of DAF-2T, and only the DAPI counterstain (gray) was observed in the merged control confocal images. The expression of DAF-2T was also studied by flow cytometry. Using LPS and NO donor DETA NONOate as positive controls for this experiment, FIG. 3C shows that the percent of DAF-2T positive live cells incubated with COMP-4 was upregulated with respect to untreated controls, and expression was similar to the level achieved by treatment with LPS and DETA NONOate.

The same effect of COMP-4 on intracellular NO production was observed using another intracellular NO detection probe, DAX-J2 Orange (FIG. 3D). Not unexpectedly, the addition of L-NAME to COMP-4 significantly reduced the expression of DAX-J2 Orange live cells by 46% when compared to COMP-4 alone.

### 2.2 COMP-4 Increases eNOS and iNOS Expression in HUAEC Cells

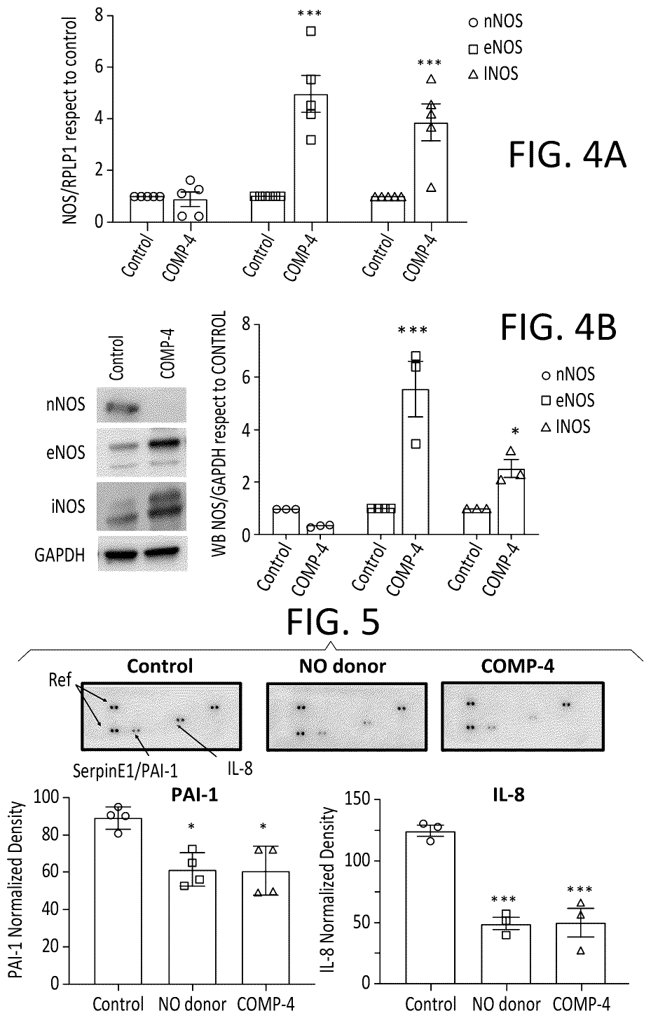

To determine whether the increase in NO production and cGMP expression observed with COMP-4 within the HUAEC could be due to changes in the expression of any of the three endogenous nitric oxide synthases, real-time RT-PCR and western blots were performed on these cells treated with or without COMP-4. FIG. 4A shows that COMP-4 significantly increased eNOS mRNA expression by 4.4-fold (p<0.01) and iNOS mRNA expression by 3.9-fold (p=0.0102) with respect to untreated controls. The expression of nNOS was not changed by COMP-4.

The protein concentrations for the three NOS enzymes were also studied by western blot. FIG. 4B shows that treatment with COMP-4 increased the expression of eNOS by 5-fold and iNOS by 2.5-fold; nNOS expression was unchanged.

### 2.3 COMP-4 Reduces Cytokine Expression in HUAEC

To determine whether COMP-4, by increasing NO production, can act as an anti-inflammatory/antifibrotic compound by modulating the expression of cytokines, the level of various cytokines released in the cell culture media in the presence or absence of COMP-4 was studied. FIG. 5 shows that after treatment of HUAEC with COMP-4 for 24 hours, there was a decrease in the expression of PAI-1 and IL-8 compared to untreated controls. The NO donor, DETA NONOate, used as a positive control for NO release, showed a similar expression profile as that for COMP-4.

### 2.4 COMP-4 Prevents Endothelial Dysfunction Induced by H₂O₂

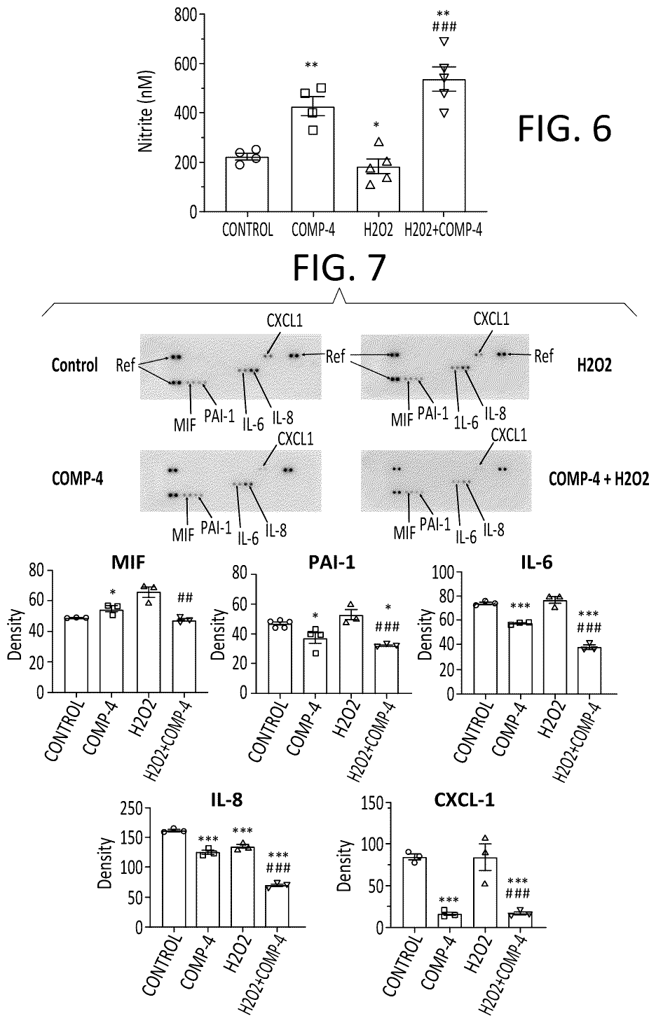

Since H₂O₂ is considered the primary source of endogenous ROS and has been extensively used to induce endothelial dysfunction in vitro (Han, R., Tang, F., Lu, M., Xu, C., Hu, J., Mei, M., & Wang, H. (2017). Astragalus polysaccharide ameliorates H₂O₂-induced human umbilical rein endothelial cell injury. Molecular Medicine Reports, 15, 4027-4034; Zheng Z, Wang M, Cheng C, Liu D, Wu L, Zhu J, Qian X. Ginsenoside Rb1 reduces H2O2 induced HUVEC dysfunction by stimulating the sirtuin 1/AMP activated protein kinase pathway. Mol Med Rep. 2020 July; 22(1):247-256. doi: 10.3892/mmr.2020.11096. Epub 2020 Apr. 28. PMID: 32377712; PMCID: PMC7248484.20,21), Applicant further investigated whether COMP-4, by increasing NO production, can improve such H₂O₂-induced endothelial dysfunction in HUAEC. FIG. 6 shows that H₂O₂ decreases nitrite formation while co-incubation of H₂O₂ with COMP-4 increases nitrite formation by 3-fold with respect to H₂O₂ alone.

In addition, to further test the hypothesis that COMP-4 can attenuate endothelial dysfunction caused by H₂O₂, the cytokine profiles in cell lysates were studied. FIG. 7 shows that H₂O₂ increased the expression of IL-6, IL-8, MIF, PAI-1, and CXCL-1/GRO, while the co-incubation of H₂O₂ with COMP-4 decreased cytokine expression, similar to the levels achieved with COMP-4 alone.

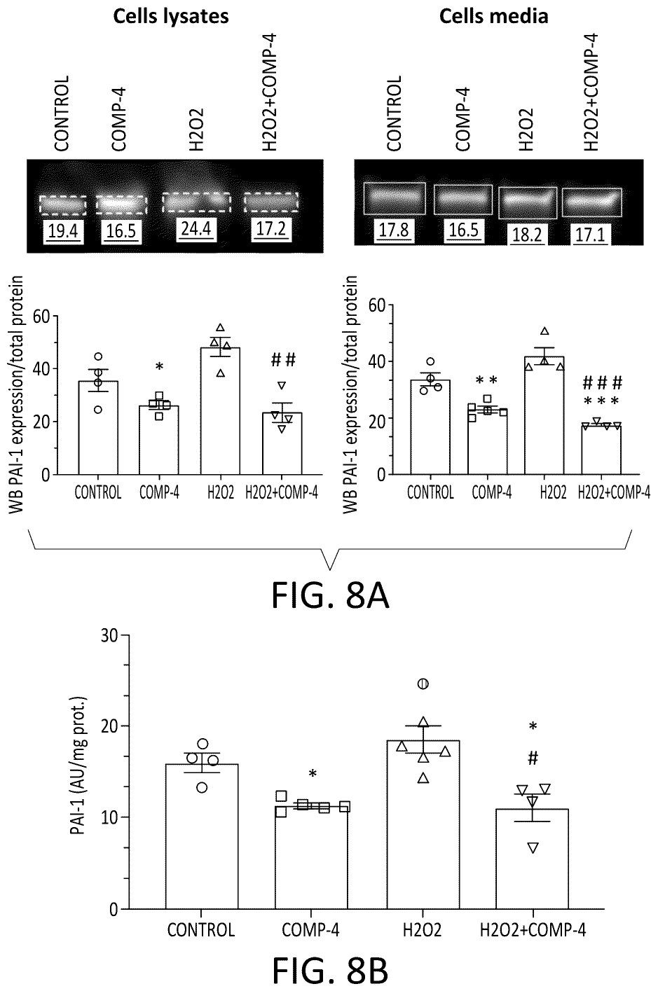

Applicant further investigated the expression of PAI-1 due to its critical role in atherothrombotic diseases, coronary artery disease, and myocardial infarction. Jung R G, Motazedian P, Ramirez F D, Simard T, Di Santo P, Visintini S, Faraz M A, Labinaz A, Jung Y, Hibbert B. Association between plasminogen activator inhibitor-1 and cardiovascular events: a systematic review and meta-analysis. Thromb J. 2018 Jun. 5; 16:12. doi: 10.1186/s12959-018-0166-4. PMID: 29991926; PMCID: PMC5987541. The reduction of the expression of PAI-1 was corroborated by WB in cell lysates and the media. FIG. 8A shows that COMP-4 treatment reduced the expression of PAI-1 in the cell lysate by 32% (p=0.0063) and in the media by 32% p=0.0292). Moreover, the expression of PAI-1 was upregulated by H₂O₂ and down-regulated by the co-incubation of COMP-4 with H₂O₂. The same results were observed by measuring the secreted PAI-1 activity in FIG. 8B. A reduction of PAI-1 activity by 42% (p=0.092) was observed after the co-incubation of H₂O₂ with COMP-4.

The present study demonstrates that in human arterial endothelial cells, the nutraceutical composition of the present invention, COMP-4, is capable of activating the eNOS/iNOS-NO-cGMP pathway, leading to an increase in the production and bioavailability of NO, as well as protection of endothelial cells from H₂O₂-induced injury.

Increases in NO and cGMP in human endothelial cells appear to be due to COMP-4's activation of eNOS and iNOS but not nNOS as evidenced by the increase in both the mRNA and protein content of the eNOS and iNOS enzymes. This was further confirmed by the observation that L-NAME, a non-selective NOS inhibitor, as well as L-NIL, a specific inhibitor of iNOS, are both capable of blocking cGMP formation by COMP-4.

Previous work has shown that NO has an anti-inflammatory effect through downregulation of pro-inflammatory cytokines. Thomassen M J, Buhrow L T, Connors M J, Kaneko F T, Erzurum S C, Kavuru M S. Nitric oxide inhibits inflammatory cytokine production by human alveolar macrophages. Am J Respir Cell Mol Biol. 1997 September; 17(3):279-83. doi: 10.1165/ajrcmb.17.3.2998m. PMID: 9308913. In the present study, COMP-4 treatment was shown to abrogate H₂O₂-induced expression of MIF, IL-6, IL-8, and CXCL1, suggesting that COMP-4 can reduce inflammation, likely by increasing NO availability. The mechanism by which COMP-4 acts to reduce cytokine expression remains unclear, since the release of pro-inflammatory cytokines is regulated in part by transcription factors such as nuclear factor-xB (NF-ϰB). Edelman D A, Jiang Y, Tyburski J G, Wilson R F, Steffes C P. Cytokine production in lipopolysaccharide-exposed rat lung pericytes. J Trauma. 2007 January; 62(1):89-93. doi: 10.1097/TA.0b013e31802dd712. PMID: 17215738. COMP-4 may act directly or indirectly through NO release in activating NF-ϰB or through other mediators.

COMP-4 treatment also appeared to attenuate H2O₂-induced endothelial dysfunction by modulating nitrite formation, a footprint of NO production. This suggests that COMP-4's effect on eNOS and iNOS expression may play a cytoprotective role by increasing NO production and bioavailability. As further evidence of a protective role for COMP-4, treatment of cells with COMP-4 reduced the expression and activity of PAI-1 in the setting of H₂O₂-induced endothelial injury. PAI-1 is mainly produced by the endothelium and has a multifaceted role in inflammation, oxidative stress, fibrosis, and macrophage adhesion/migration. Jung R G, Motazedian P, Ramirez F D, Simard T, Di Santo P, Visintini S, Faraz M A, Labinaz A, Jung Y, Hibbert B. Association between plasminogen activator inhibitor-1 and cardiovascular events: a systematic review and meta-analysis. Thromb J. 2018 Jun. 5; 16:12. doi: 10.1186/s12959-018-0166-4. PMID: 29991926; PMCID: PMC5987541; Edelman D A, Jiang Y, Tyburski J G, Wilson R F, Steffes C P. Cytokine production in lipopolysaccharide-exposed rat lung pericytes. J Trauma. 2007 January; 62(1):89-93. doi: 10.1097/TA.0b013e31802dd712. PMID: 17215738; Yamamoto K, Takeshita K Kojima, Takamatsu J, Saito H. Aging and plasminogen activator inhibitor-1 (PAI-1) regulation: implication in the pathogenesis of thrombotic disorders in the elderly. Cardiovasc Res. 2005 May 1; 66(2):276-85. doi: 10.1016/j.cardiores.2004.11.013. Epub 2004 Dec. 8.PMID: 15820196; Liu R M. Oxidative stress, plasminogen activator inhibitor 1, and lung fibrosis. Antioxid Redox Signal. 2008 Febuary; 10(2):303-19. doi: 10.1089/ars.2007.1903. PMID: 17979497; PMCID: PMC3686819; Ghosh AK, Vaughan DE. PAI-1 in tissue fibrosis. J Cell Physiol. 2012 Febuary; 227(2):493-507. doi: 10.1002/jcp.22783. PMID: 21465481; PMCID: PMC3204398; Kwak S Y, Park S, Kim H, Lee S J, Jang W S, Kim M J, Lee S, Jang W I, Kim A R, Kim E H, Shim S, Jang H. (2021). Atorvastatin Inhibits Endothelial PAI-1-Mediated Monocyte Migration and Alleviates Radiation-Induced Enteropathy. Int J Mol Sci. 2021 Feb. 12; 22(4):1828. doi: 10.3390/ijms22041828. PMID: 33673196; PMCID: PMC7917640; Kubala M. H., Punj V., Placencio-Hickok V. R., Fang H., Fernandez G. E., Sposto R., DeClerck Y. A. Plasminogen Activator Inhibitor-1 Promotes the Recruitment and Polarization of Macrophages in Cancer. Cell Rep. 2018; 25:2177-2191.e7. doi: 10.1016/j.celrep.2018.10.082. The PAI-1 results from this study suggest that COMP-4 may modulate tissue fibrosis and remodeling even after endothelial dysfunction has already occurred. This result is consistent with Applicant's prior observation in rat cavernosal smooth muscle with long-term treatment of senescent tissue with COMP-4. Ferrini M G, Hlaing S M, Chan A, et al. Treatment with a combination of ginger, L-citrulline, muira puama and Paullinia cupana can reverse the progression of corporal smooth muscle loss, fibrosis and veno-occlusive dysfunction in the aging rat. Andrology (Los Angel). 2015 June; 4(1):132. doi: 10.4172/2167-0250.1000132. Epub 2015 May 25. PMID: 26405615; PMCID: PMC4578663.

Applicant's previous results in cavernosal smooth muscle cells showed that the complete COMP-4 compound rather than any of the individual four components had the most profound effect in modulating each one of the specific steps within the iNOS-cGMP pathway, as well as in reducing oxidative stress markers in the penile corpora cavernosa and as such is presumed to be the same with respect to the effect of COMP-4 on endothelial cells. Ferrini M G, Abraham A, Graciano L, Nguyen S, Mills J N, Rajfer J. Activation of the iNOS/NO/cGMP pathway by Reractin® in human corporal smooth muscle cells. Transl Androl Urol. 2021 July; 10(7):2889-2898. doi: 10.21037/tau-21-11. PMID: 34430391; PMCID: PMC8350259; Rajfer R A, Kilic A, Neviaser A S, Schulte L M, Hlaing S M, Landeros J, Ferrini M G, Ebramzadeh E, Park S H. Enhancement of fracture healing in the rat, modulated by compounds that stimulate inducible nitric oxide synthase: Acceleration of fracture healing ria inducible nitric oxide synthase. Bone Joint Res. 2017 Febuary; 6(2):90-97. doi: 10.1302/2046-3758.62.BJR-2016-0164.R2. PubMed PMID: 28188129; PubMed Central PMCID: PMC5331177; Nguyen, S., Castellanos, K. A., Abraham, A. et al. Reduction of oxidative stress markers in the corpora cavernosa and media of penile dorsal artery in middle-aged rats treated with COMP-4. Int J Impot Res 33, 67-74 (2021).

Taken together, the present study shows that COMP-4, an off-the-shelf nutraceutical product consisting of ginger, L-citrulline, muira puama, and Paullinia cupana, may have a protective role against arterial endothelial dysfunction. This effect is accomplished through activation of the eNOS/iNOS-NO-cGMP pathway and ameliorates the pro-inflammatory milieu induced by H₂O₂ injury. Furthermore, the effect of COMP-4 treatment on PAI-1 expression and activity in response to cell injury suggests that there may be a senolytic role for COMP-4 or other nutraceutical or herbal compounds that target NO production. COMP-4 has been studied in humans and has an innocuous side effect profile. Nguyen S, Rajfer J, Shaheen M. Safety and efficacy of daily Revactin® in men with erectile dysfunction: a 3-month pilot study. Transl Androl Urol. 2018; 7:266-273. A human subject trial to confirm efficacy in combating endothelial dysfunction appears warranted. COMP-4 plays a role in protecting the endothelial cells. The mechanism by which COMP-4 protects the endothelial cells may involve restoring the balance between NO and ROS via increasing the endothelial cell NO bioavailability and antioxidant capacity.

While the preferred embodiments have been shown and described, it will be understood that there is no intent to limit the invention by such disclosure, but rather, is intended to cover all modifications and alternate constructions falling within the spirit and scope of the invention.

1. A method for protecting human arterial endothelial cells from endothelial cell dysfunction, comprising:
administering a pharmaceutically effective amount of a composition over a sufficient period of time to stimulate NOS and protecting human arterial endothelial cells from endothelial cell dysfunction.

2. The method according to claim 1, wherein the composition comprises ginger or a ginger derivative, Muira puama, Paullinia cupana, and L-arginine and/or L-citrulline.

3. The method according to claim 2, wherein the composition comprises 250 mg to 2 g ginger or ginger derivative.

4. The method according to claim 2, wherein the composition comprises 10 mg to 3 g of L-arginine, L-citrulline, or a mixture of L-arginine and L-citrulline.

5. The method according to claim 2, wherein the composition comprises 10 mg to 2 g of L-arginine, L-citrulline, or a mixture of L-arginine and L-citrulline.

6. The method according to claim 2, wherein the composition comprises 100 mg to 3 g of Muira puama.

7. The method according to claim 2, wherein the composition comprises 500 mg to 1.5 g of Muira puama.

8. The method according to claim 2, wherein the composition comprises at least 250 mg of Paullinia cupana.

9. The method according to claim 2, wherein the composition comprises 500 mg of Paullinia cupana.

10. The method according to claim 2, wherein the composition comprises 250 mg to 2 g of ginger or ginger derivative, 250 mg to 2 g of L-arginine, L-citrulline, or mixture of L-arginine and L-citrulline, 500 mg to 1.5 g of Muira puama, and 500 mg of Paullinia cupana.
